(19) United States
(12) Patent Application Publication
(10) Pub. No.: US 2009/0326340 A1
(43) Pub. Date: Dec. 31, 2009

(54) Patient Monitor Alarm System And Method

(76) Inventors: Hui Wang, San Ramon, CA (US); Scott Amundson, Oakland, CA (US)

Correspondence Address: NELLCOR PURITAN BENNETT LLC;ATTN: IP LEGAL, 6135 Gunbarrel Avenue, Boulder, CO, 80301, US

(21) Appl. No.: 12/165,025

(22) Filed: Jun. 30, 2008

(51) Int. Cl. A61B 5/00; G08B 23/00

(52) U.S. Cl. 600/301; 340/573.1

(57) ABSTRACT

Provided herein is a patient monitoring alarm escalation system and method, according to embodiments, which may include a medical device configured to measure physiological data received via a patient monitor configured to initiate an alarm in response to predefined measurements of the physiological data. The medical device is configured to communicate with a medical station and escalate an alarm if an alarm acknowledgement mechanism at the medical station is not activated.

### BACKGROUND

The present disclosure relates generally to medical devices, and, more particularly, systems and methods for generating and delivering a patient alarm to attending personnel and/or to the patient

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Patient monitors include medical devices that facilitate observation of patient physiological data. For example, a typical patient monitor detects and displays a patient's vital signs continually. This improves patient care by facilitating continuous supervision of a patient without continuous attendance by a human observer (e.g., a nurse or physician). Typically, patient monitors include alarm systems that provide audible and/or visual indications of certain predefined conditions. For example, some patient monitors include alarms that are triggered based on physiological conditions (e.g., high and low patient heart rate thresholds, arterial oxyhemoglobin saturation) or status indicators for the monitor itself (e.g., power loss). These alarms further facilitate supervision of patients and improve patient care by providing caregivers with warnings concerning certain monitored conditions. Generally, such alarms remain in an alarm state until acknowledged by a user. For example, an audible alarm for a patient's abnormal systolic condition may continue to sound until a user presses an acknowledge button that silences the alarm and indicates that the alarm has been acknowledged.

Although presently known monitoring systems and methods are generally adequate to safeguard the health of the patient various drawbacks nevertheless exist. For example, in order to avoid disturbing the patient during sleep periods, an alarm device located close to the patient may be turned off, or otherwise overridden, while an alarm device located at a central monitoring location remains on. Consequently, if the attending medical personnel are absent from the central monitoring location, or otherwise fail to respond to a generated alarm state, the alarm may not be heeded.

### BRIEF DESCRIPTION OF THE DRAWINGS

Advantages of the disclosure may become apparent upon reading the following detailed description and upon reference to the drawings in which:

FIG. 1 is a block diagram of a system in accordance with an embodiment;

FIG. 2 is a block diagram of a patient monitor for providing alarms in accordance with an embodiment;

FIG. 3 is flowchart of a method of generating escalated alarms in accordance with an embodiment;

FIG. 4 is flowchart of a alternative method of generating escalated alarms in accordance with an embodiment; and

FIG. 5 is an alarm display screen in accordance with an embodiment.

### DETAILED DESCRIPTION

One or more embodiments will be described below. In an effort to provide a concise description of embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Provided herein are embodiments of systems and methods for generating and tracking alarms and alarm conditions associated with patient care. In certain patient settings, alarms may be silenced or left unattended (i.e., unacknowledged) at the bedside monitor of a patient. Such alarms may be sent forward to a central management station, such as a nurse's station, either at the time the bedside monitor alarm is generated, or after a certain amount of time has passed while the beside alarm remains unacknowledged. The central management station provides certain redundancy in management of alarm conditions. However, as with bedside alarms, alarms may be left unattended while other, more pressing, patient conditions are of concern. The present techniques provide additional alarm escalations for unattended alarms to alert caregivers of the alarm in an escalating manner over time. In an embodiment, additional alarms may be sent to wireless devices carried by caregivers if lower stages of the alarm escalation are unattended. In an embodiment, the alarm escalation may involve triggering an audible alarm at the bedside, so that the bedside alarm may be audible to the patient or bedside nurse. In addition, the present techniques may allow caregivers to track the alarm messages so that each alarm escalation carries an indicator of how far the escalation has risen, which may provide more information about how the urgency of an unattended low-level alarm.

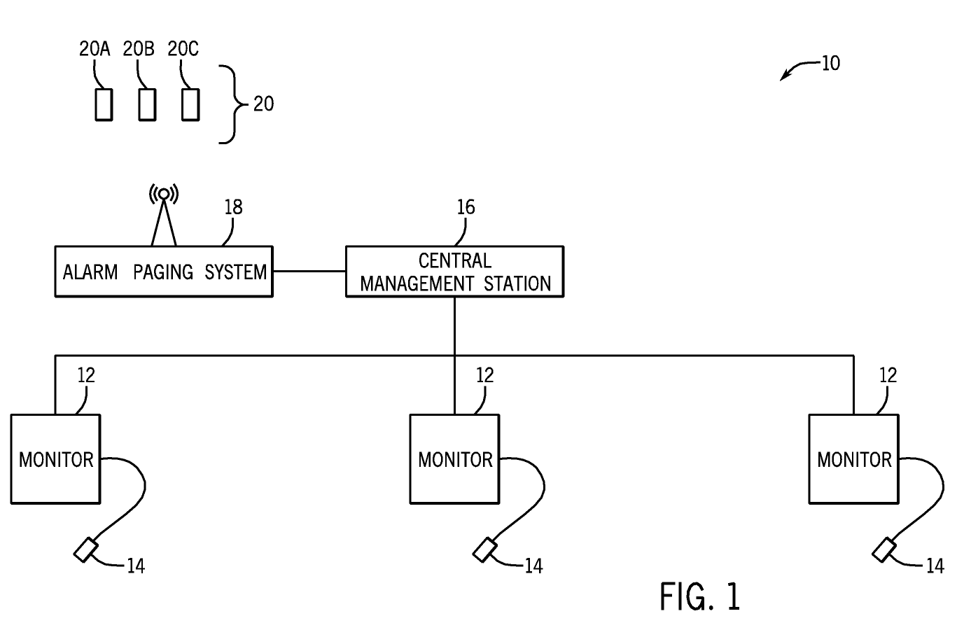

FIG. 1 is a block diagram of a monitoring system in accordance with an embodiment. Specifically, FIG. 1 illustrates a monitoring system 10 capable of generating alarms related to various conditions, such as patient conditions or certain conditions associated with the device (e.g., low battery). The system 10 may include a plurality of patient monitors 12 (which may be multiple monitors associated with a single patient or multiple monitors associated with multiple patients) that collect data through sensors 14. Suitable monitors may include pulse oximetry monitors, as well as any suitable blood pressure monitors, EKG monitors, sleep apnea monitors, multiparameter monitors, or other types of patient monitors. The monitors 12 may be networked to a central management station 16 (e.g., a personal computer). An exemplary central management station may include a Nellcor® Oxinet® III central station and paging system, from Nellcor Puritan Bennett LLC. The patient monitor 12 and the central management station 16 may include respective alarm systems that may be housed within the devices or that may exist as separate structures in an embodiment. The system 10 may also include an alarm paging system 18 that is operable to communicate with one or more wireless and mobile pagers 20. This monitoring system 10 facilitates monitoring multiple patients in, for example, a hospital or clinic. The monitoring system 10 may be networked with network cables. However, in an embodiment, wireless communication is utilized.

Each of the patient monitors 12 may include a sensing device 14 (e.g., a pulse oximetry sensor) for measuring patient physiological data. Additionally, each of the monitors 12 or the central management station 16 may be configured to generate an alarm based on predefined physiological data values or conditions relating to such values. For example, an alarm may be activated when a patient's oxygen saturation has been at a certain level for a predefined amount of time.

When alarm conditions are detected, the system 10 may emit alarm signals from any one of the monitors 12, the central management system 16, and/or the alarm paging system 98. Further, if the alarm is not acknowledged, the monitoring system 10 may escalate the alarm. For example, in an embodiment, a primary alarm signal may be generated at a single bedside patient monitor 12. In an embodiment, a visual or text alarm may be generated at the bedside and an audio alarm may be generated/logged simultaneously at the central management station 16. If this primary alarm is not acknowledged within a predefined amount of time, a second alarm may be sent to a wireless device 20 that is carried by a caregiver. If the second alarm remains unacknowledged for a predefined amount of time (e.g., half of the time allotted to acknowledge the primary alarm), a third alarm may be sent to an additional wireless device 20 and so forth. Additionally, the urgency level of each pager alarm may be increased. For example, the pagers may beep or vibrate with a higher amplitude and/or frequency. In an embodiment, for patients who are being monitored in their own homes, an alarm may be sent to a home alarm system that may be linked to an emergency response.

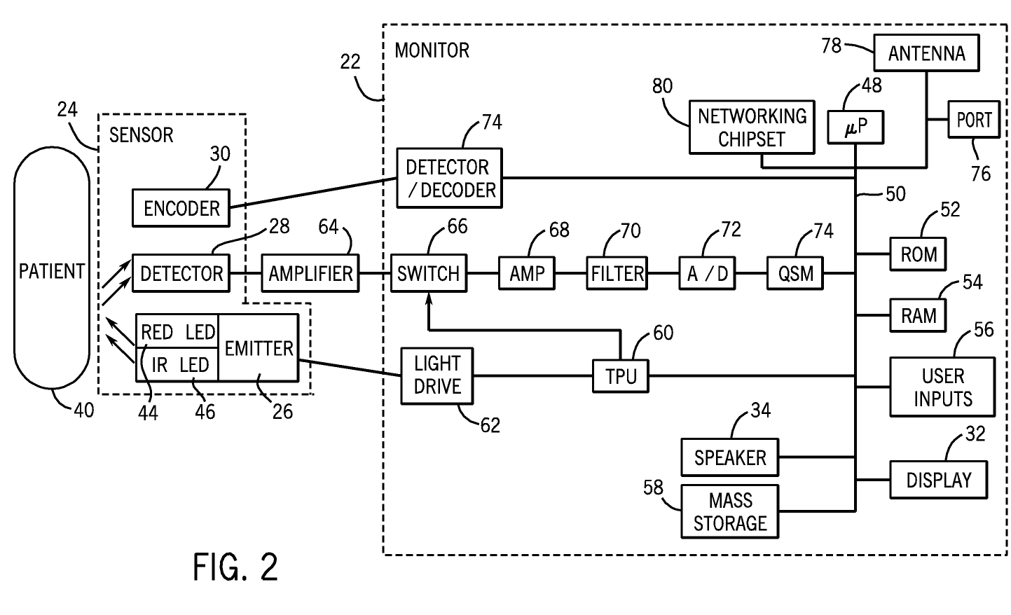

FIG. 2 is a block diagram of a patient monitor 12 of FIG. 1, such as a pulse oximeter 22 coupled to a patient 40 in according to embodiments. Examples of pulse oximeters that may be used in the implementation of the present disclosure include pulse oximeters available from Nellcor Puritan Bennett LLC, but the following discussion may be applied to other pulse oximeters and medical devices. The pulse oximeter 22 illustrated in FIG. 2 may include a sensor 24. The sensor 24 may include an emitter 26, the detector 28, and an encoder 30. It should be noted that the emitter 26 may be capable of emitting at least two wavelengths of light, e.g., RED and IR, into a patient's tissue 40. Hence, the emitter 26 may include a RED LED 44 and an IR LED 46 for emitting light into the patient's tissue 40 at the wavelengths used to calculate the patient's physiological characteristics. In certain embodiments, the RED wavelength may be between about 600 nm and about 700 nm, and the IR wavelength may be between about 800 nm and about 1000 nm. Alternative light sources may be used in other embodiments. For example, a single wide-spectrum light source may be used, and the detector 28 may be capable of detecting certain wavelengths of light. In another example, the detector 28 may detect a wide spectrum of wavelengths of light, and the monitor 22 may process only those wavelengths which are of interest. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In an embodiment, the detector 28 may be capable of detecting the intensity of light at the RED and IR wavelengths. In operation, light enters the detector 28 after passing through the patient's tissue 40. The detector 28 may convert the intensity of the received light into an electrical signal. The light intensity may be directly related to the absorbance and/or reflectance of light in the tissue 40. That is, when more light at a certain wavelength is absorbed or reflected, less light of that wavelength is typically received from the tissue by the detector 28. After converting the received light to an electrical signal, the detector 28 may send the signal to the monitor 22, where physiological characteristics may be calculated based at least in part on the absorption of the RED and IR wavelengths in the patient's tissue 40.

According to embodiments, the encoder 30 may contain information about the sensor 24, such as what type of sensor it is (e.g., whether the sensor is intended for placement on a forehead or digit) and the wavelengths of light emitted by the emitter 26. This information may allow the monitor 22 to select appropriate algorithms and/or calibration coefficients for calculating the patient's physiological characteristics. The encoder 30 may, for instance, be a coded resistor which stores values corresponding to the type of the sensor 24 and/or the wavelengths of light emitted by the emitter 26. These coded values may be communicated to the monitor 22, which determines how to calculate the patient's physiological characteristics. In another embodiment, the encoder 30 may be a memory on which information may be stored for communication to the monitor 22. This information may include, for example, the type of the sensor 24, the wavelengths of light emitted by the emitter 26, and the proper calibration coefficients and/or algorithms to be used for calculating the patient's physiological characteristics. Pulse oximetry sensors capable of cooperating with pulse oximetry monitors include the OxiMax® sensors available from Nellcor Puritan Bennett LLC.

Signals from the detector 28 and the encoder 30 may be transmitted to the monitor 22. The monitor 22 generally may include one or more processors 48 connected to an internal bus 50. Also connected to the bus may be a read-only memory (ROM) 52, a random access memory (RAM) 54, user inputs 56, one or more mass storage devices 58 (such as hard drives, disk drives, or other magnetic, optical, and/or solid state storage devices), a display 32, and a speaker 34. A time processing unit (TPU) 60 may provide timing control signals to a light drive circuitry 62 that controls when the emitter 26 is illuminated and the multiplexed timing for the RED LED 44 and the IR LED 46. The TPU 60 may also control the gating-in of signals from detector 28 through an amplifier 64 and a switching circuit 66. These signals may be sampled at the proper time, depending upon which light source is illuminated. The received signal from the detector 28 may be passed through an amplifier 68, a low pass filter 70, and an analog-to-digital converter 72. The digital data may then be stored in a queued serial module (QSM) 74 for later downloading to the RAM 54 or mass storage 58 as the QSM 74 fills up. In one embodiment, there may be multiple separate parallel paths having the amplifier 68, the filter 70, and the A/D converter 72 for multiple light wavelengths or spectra received.

Signals corresponding to information about the sensor 24 may be transmitted from the encoder 30 to a decoder 74. The decoder 74 may translate these signals to enable the processor 48 to determine the proper method for calculating the patient's physiological characteristics, for example, based generally on algorithms or look-up tables stored in the ROM 52 or mass storage 58. In addition, or alternatively, the encoder 30 may contain the algorithms or look-up tables used by the processor 48 for calculating the patient's physiological characteristics.

According to embodiments, the monitor 22 may also include one or more mechanisms to facilitate communication with other devices in a network environment, such as the central management station 16 (see FIG. 1). For example, the monitor 22 may include a network port 76 (such as an Ethernet port) and/or an antenna 78 by which signals may be exchanged between the monitor 14 and other devices on a network, such as servers, routers, workstations and so forth. In some embodiments, such network functionality may be facilitated by the inclusion of a networking chipset 80 within the monitor 22 though in other embodiments the network functionality may instead be provided by the processor(s) 48. In an embodiment, the central management station 16 may communicate with the monitor 22 via such networking devices as provided. As a result of such communication, the central management station 16 may provide instructions to be executed by processor 48 that involve triggering audible or other escalated alarms.

According to embodiments, the pulse oximeter 22 may also be configured to provide alarms under certain conditions. Alarm conditions may be designated by set points or by designating patterns of values (e.g., patterns in an SpO2 trend) or limits that can be entered via adjustment buttons. For example, a user can input a certain set point (e.g., 103 degrees Fahrenheit, blood oxygen level of 97%) that creates an alarm condition when crossed by actual patient data (e.g., actual patient temperature, actual blood oxygen level), or when processed values or patterns of values are detected. The patient monitor 22 may detect alarm conditions with an alarm system that may involve instructions executed on processor 48 that compare designated set points with actual patient data received from a sensor 24 via a cable connection port that is configured to communicatively couple with the sensor 24. For example, in some embodiments, the alarm system employs SatSeconds by Nellcor Puritan Bennett, incorporated, to detect alarms and manage nuisance alarms. SatSeconds may include alarming based on an integral of time and depth of a desaturation event. It should be noted that, in some embodiments, alarms are visually and/or haptically indicated in addition to or instead of being audibly indicated. Indeed, alarms may be indicated to alert any of a caregiver's senses (e.g., sight, touch, and hearing). These alternative sensory indications (e.g., alarm lights and vibrating pagers) are additional tools with which a user's attention can be directed to an alarm condition. For example, the pulse oximeter 22 may include a display 32, such as a liquid crystal display (LCD), that visibly displays alarm indications and other information. In one embodiment, the display 32 is configured to visually communicate patient physiological data (e.g., oxygen saturation percentage, pulse amplitude, pulse rate) and alarms in the form of numeric data, textual data, and/or graphical data (e.g., plethysmographic waveforms and/or alarm icons). The display 32 may also be configured to display equipment status indicators such as an on/off indication, a power indication depending on whether a power cord is receiving power, and/or other equipment status indicators that may also trigger certain alarms. In one embodiment, the display 32 is used to visually confirm values entered while configuring aspects of the pulse oximeter 22 (e.g., providing set points for alarms). In an embodiment, certain alarm conditions may be set to avoid triggering an audible signal to avoid disturbing the patient. In such an embodiment, upon escalation, the alarm silence may be overridden when the alarm is escalated. In one embodiment, certain alarms may be designated as being of sufficient urgency that previous settings regarding alarm silencing may be overridden. Such alarms may also be associated with downstream alarm messages being sent directly to a crash cart or urgent care team.

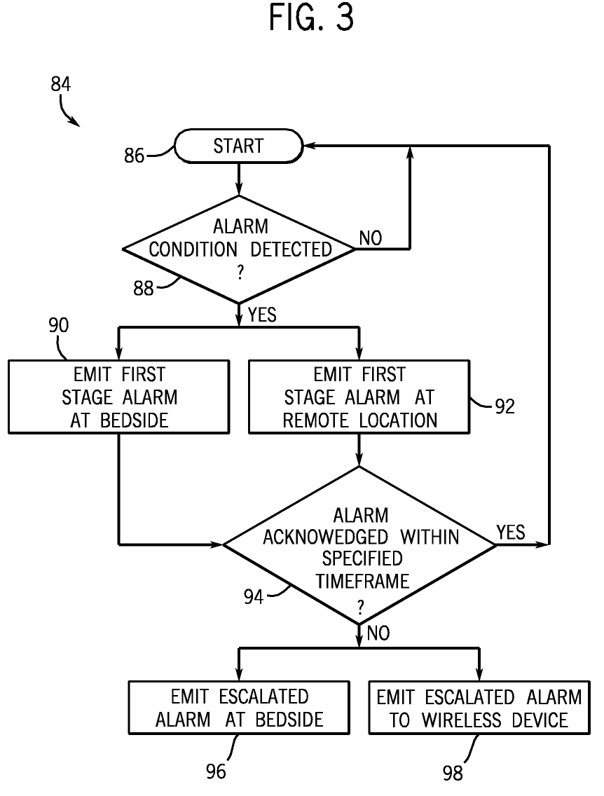

FIG. 3 is a block diagram of a method 84 for providing patient monitor alarms in accordance with an embodiment. The method 84 can be implemented with a single alarm indicator or multiple alarm indicators. For example, embodiments may use speakers, pagers, visual indicators, and/or haptic devices to provide the alarms. The method 84 begins at block 86 and proceeds to block 88, which is a decision block regarding whether an alarm condition has been detected at a patient monitor. If an alarm condition has not been detected, the method returns to the start (block 86). If an alarm condition has been detected, a first stage bedside alarm signal is emitted by one or multiple alarm indicators (e.g., speaker 34, display 32) in block 90. The alarm signal may include a tone emitted from a speaker, a light emitted from a display and so forth. In an embodiment, the first stage bedside alarm is inaudible, which may be an appropriate setting for when a patient is asleep. In addition, at block 92 a first stage alarm signal is emitted at a remote location, such as a central management station 16. The first stage alarm at the remote location may be any suitable alarm, and may not necessarily be the same as the first stage bedside alarm.

In an embodiment, after an alarm has been initiated (block 88), the method 84 begins determining whether the alarm condition still exists and/or whether the alarm signal has been acknowledged, as illustrated by block 94. Specifically, block 94 is a decision block regarding whether a user has provided confirmation that the alarm condition has been recognized or acknowledged at the bedside and/or remote location within a predetermined timeframe. This timeframe may be specific to and appropriate for each individual alarm condition. If one or both of the alarms has been acknowledged, the method may return to start 86. Such an indication of acknowledgement may be provided by, for example, depressing an alarm silence button. If the alarm condition has been acknowledged, an alarm timer may be reset or canceled and an alarm silence timer may be initiated. In some embodiments, the alarm silence timer is not utilized. For example, in some embodiments, once a specific alarm is acknowledged, the same alarm condition will not initiate the primary alarm again, thus eliminating potentially redundant alarms. In other words, in such embodiments, the same alarm condition may not cause repeated alarm signals to be periodically emitted after acknowledgement when the alarm silence timer expires.

In an embodiment if the alarm has not been acknowledged within the specified timeframe, the method 84 may emit escalated alarms at one or more locations. As shown, an alarm may be escalated at the bedside location in block 96 as well as sent to any suitable wireless or paging device in block 98. This not only serves to increase awareness but also provides redundancy. Accordingly, if an alarm in a central management station is not acknowledged, an escalated alarm at the bedside, such as a loud speaker tone may either alert the patient to the condition or may alert a caregiver. This allows the first stage alarm at the bedside to be silent, in order to decrease nuisance noises at the bedside.

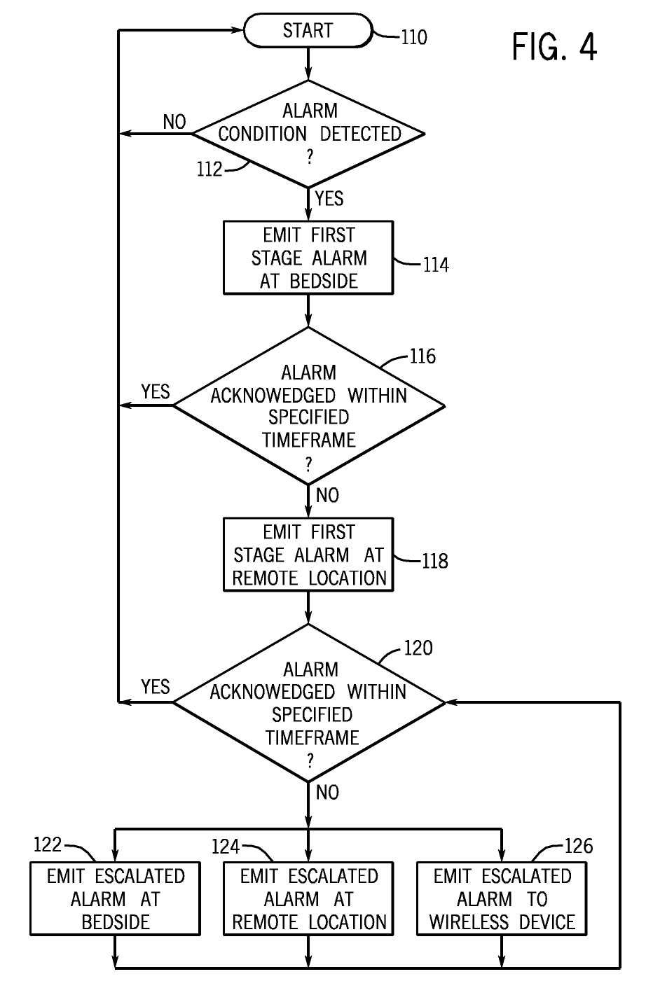

FIG. 4 is a block diagram of an alternative method 108, according to an embodiment. The method 108 begins at block 110 and proceeds to block 112, which is a decision block regarding whether an alarm condition has been detected at a patient monitor. If an alarm condition has not been detected, the method returns to the start (block 108). If an alarm condition has been detected, a first stage bedside alarm signal is emitted by one or multiple alarm indicators (e.g., speaker 34, display 32) in block 114. After an alarm has been initiated (block 114), the method 108 begins determining whether the alarm condition still exists and/or whether the alarm signal has been acknowledged in block 116, a decision block for whether a user has provided confirmation that the alarm condition has been recognized or acknowledged at the bedside within a predetermined timeframe. If the bedside alarm has been acknowledged, the method may return to start 110. In the bedside alarm is not acknowledged, in block 118 a first stage alarm signal is emitted at a remote location, such as a central management station 16. The first stage alarm at the remote location may be any suitable alarm, and may not necessarily be the same as the first stage bedside alarm.

In embodiments, if the first stage bedside alarm and the first stage remote alarm have not been acknowledged within the specified timeframe, the method 108 may emit escalated alarms at one or more locations. As shown, an alarm may be escalated at the bedside location in block 122, the remote location in block 124 as well as sent to any suitable wireless or paging device in block 126. If these escalated alarms are not acknowledged, the method 108 may continue to escalate the level of the alarms until the alarm condition is no longer in place or until the alarm has been acknowledged at one or more locations. Further, in an embodiment (not shown), the central management station 16 can send further alarms to additional wireless devices if the initial wireless device does not acknowledge the alarm.

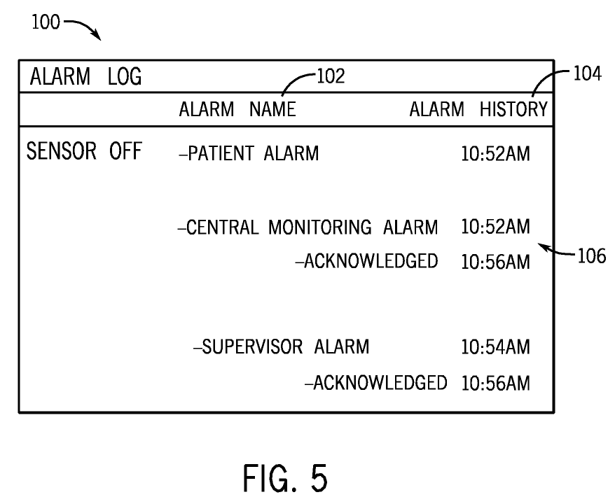

FIG. 5 is an exemplary alarm history display screen 100 that may be displayed on a patient monitor 12 or a central management station 16, according to an embodiment. The screen may include a log of any triggered alarms. As shown, the screen 100 may include a field for alarm type 102 and a field for the alarm history 104. The alarm history may include alarm data 106 such as trigger times as well as alarm acknowledgement history. In addition, the alarm data 106 may include a history of the level of escalation as well as details regarding the locations and/or devices to which the alarm messages were sent. Alarm data 106 may also include patient-specific information such as patient identification information and/or patient location information. The alarm data 106 may also include real-time oxygen saturation and/or pulse data as well as oxygen saturation and pulse rate trend information.

While only certain features have been illustrated and described herein, many modifications and changes will occur to those skilled in the art. It is, therefore, to be understood that the appended claims are intended to cover all such modifications and changes as fall within their true spirit.

1. An alarm system compnsing:
a physiological monitor capable of triggering an inaudible first alarm in response to an alarm condition; and
a station located remotely from the physiological monitor, wherein the station is capable of receiving an input from the physiological monitor and triggering a second alarm in response to the alarm condition, and wherein when the second alarm is not acknowledged, the station is capable of sending an output to the physiological monitor to initiate an escalated audible alarm at the physiological monitor.

2. The system of claim 1, wherein the second alarm is capable of being acknowledged by an operator input before expiration of a timer.

3. (canceled)

4. (canceled)

5. (canceled)

6. The system of claim 1, wherein the first alarm and the second alarm are triggered simultaneously.

7. The system of claim 1, wherein the station located remotely from the physiological monitor is part of a central patient management system.

8. A physiological monitor comprising:
a processor programmed to:
trigger an inaudible first alarm in response to an alarm condition;
send an output to a remotely located station, wherein the output triggers a second alarm in response to the alarm condition; and
receive an input from the remotely located station, wherein the input causes physiological monitor to initiate an escalated audible alarm.

9. The physiological monitor of claim 8, wherein the processor is programmed to send an output to a wireless device when the escalated alarm is not acknowledged.

10. (canceled)

11. (canceled)

12. (canceled)

13. The physiological monitor of claim 8, wherein the first alarm and the second alarm are triggered simultaneously.

14. A method, comprising:
Triggering an inaudible first alarm in response to an alarm condition;
triggering a second alarm remote from the first location in response to the alarm condition; and
escalating, through an audible alarm, the first alarm if neither of the first or second alarms are acknowledged with a certain time.

15. (canceled)

16. A system comprising:
a processor programmed to
trigger an inaudible first alarm in response to an alarm condition;
send an output to a remotely located station, wherein the output triggers a second alarm in response to the alarm condition: and
receive an input from the remotely located station, wherein the input causes the physiological monitor to initiate an audible escalated alarm.
a memory capable of storing alarm data associated with the alarms, wherein the alarm data comprises an alarm type and a time that the alarm was triggered; and
a display capable of displaying the alarm data.

17. The system of claim 16, wherein the system is part of a physiological monitor or a central management system.

18. The system of claim 16, wherein the alarm data comprises information about a level of escalation of the alarm.

19. The system of claim 16, wherein the alarm data comprises information about an acknowledgement of the alarm.

20. The system of claim 16, wherein the processor is programmed to send the alarm data to one or more wireless devices.
